(19) United States
(12) Patent Application Publication
(10) Pub. No.: US 2012/0238927 A1
(43) Pub. Date: Sep. 20, 2012

(54) PATELLA SUPPORT

(75) Inventor: Pang-Ching CHIANG, Taipei City (TW)

(73) Assignee: PLUS MEDITECH CO., LTD., Tainan City (TW)

(21) Appl. No.: 13/048,392

(22) Filed: Mar. 15, 2011

(51) Int. Cl. A61F 5/00

(52) U.S. Cl. 602/26

(57) ABSTRACT

A patella support includes: a strip adapted to loop around a knee joint and including a patella bracing portion; and a patella protective member attached to an inner side of the strip for preventing a lateral movement of a patella of the knee joint. The patella protective member includes: an intermediate portion having an outer face fastened to an inner surface of the patella bracing portion, and an inner face adapted to face the patella; two lateral portions extending oppositely from two ends of the intermediate portion, and fastened to the patella bracing portion; and a resilient cushion pad attached to the intermediate portion and adapted to cushion the patella.

### BACKGROUND OF THE INVENTION

1. Field of the Invention

This invention relates to a protector, more particularly to a patella support.

2. Description of the Related Art

An abnormal motion of a patella, which is a bone located at an anterior port ion of a knee joint, may result in injuries of the knee joint during exercise. A patella support is developed for preventing the patella from being hurt due to an undue lateral movement or weight load.

As shown in FIG. 1, a conventional patella support 1 is an elastic sleeve having an annular pad 11. In a use state, the patella support 1 should be put on and moved to be sleeved on a knee joint, and then the annular pad 11 is moved to surround and abut against the patella for preventing a lateral movement of the patella.

However, since the stretching degree of the patella support 1 cannot be adjusted to fit the shape of the user's patella or to depend on kinds of exercises, the patella support 1 may be sleeved on the knee joint in an overly tight or too loose manner and thus, the patella is still likely to be injured due to overly lateral movement.

### SUMMARY OF THE INVENTION

Therefore, an object of the present invention is to provide a patella support that can overcome the aforesaid drawbacks associated with the prior art.

Accordingly, a patella support of the present invention comprises:

a strip adapted to loop around a knee joint and including a patella bracing portion; and

a patella protective member attached to an inner side of the strip for preventing a lateral movement of a patella of the knee joint, and including:

an intermediate portion having an outer face fastened to an inner surface of the patella bracing portion, and an inner face adapted to face the patella;

two lateral portions extending oppositely from two ends of the intermediate portion, and fastened to the patella bracing portion; and

a resilient cushion pad attached to the intermediate port ion and adapted to cushion the patella

### BRIEF DESCRIPTION OF THE DRAWINGS

Other features and advantages of the present invent ion will become apparent in the following detailed description of the preferred embodiment of the invention, with reference to the accompanying drawings, in which:

FIG. 1 is a perspective view of a conventional patella support;

FIG. 2 is an exploded elevation view of the preferred embodiment of a patella support according to the present invention;

FIG. 3 is a rear view of FIG. 2;

FIG. 4 is a perspective view of the patella support of this invention when the patella support is in a use state; and

FIG. 5 is the same view as FIG. 4 but illustrating the patella support at a different angle of view.

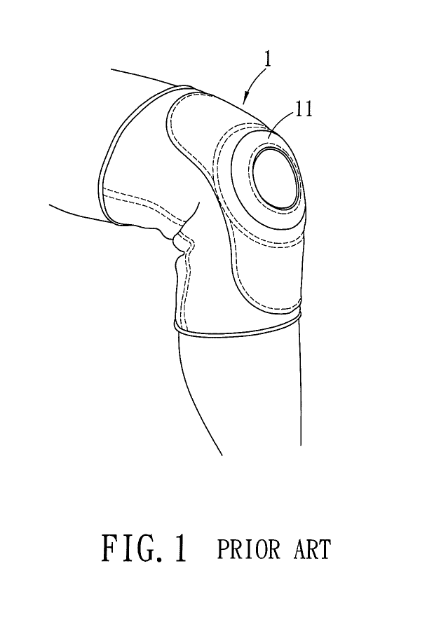

### DETAILED DESCRIPTION OF THE PREFERRED EMBODIMENT

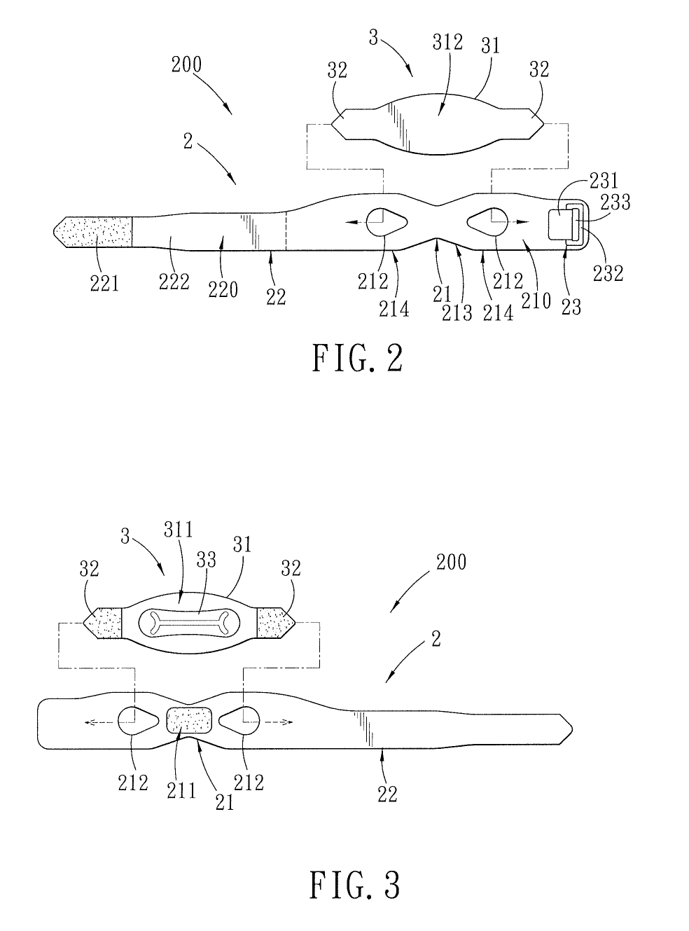

Referring to FIGS. 2 to 5, the preferred embodiment of a patella support 200 according to the present invention comprises a strip 2 and a patella protective member 3.

The strip 2 is adapted to loop around a knee joint and includes a patella bracing portion 21, a strip extension 22, and a fastener 23. The patella bracing portion 21 is formed with two holes 212. The strip extension 22 extends from one side of the patella bracing portion 21. The fastener 23 is disposed on the other side of the patella bracing portion 21 opposite to the strip extension 22.

Preferably, the patella bracing portion 21 has a narrow mid part 213, and two enlarged parts 214 on two opposite sides of the narrow mid part 213. The two holes 212 are disposed in the enlarged parts 214, respectively.

An outer surface 220 of the strip extension 22 includes two parts 221, 222 that are connected to each other to form an adjustable loop of the strip 2 by means of Velcro fasteners. That is to say, one of the parts 221, 222 has a “loop” side, and the other of the same has a “hook” side. The strip extension 22 is connected adjustably to the fastener 23 such that the strip 2 forms the adjustable loop.

The fastener 23 includes a buckle 232 attached to an outer surface 210 of the patella bracing portion 21 through a strip loop 231. The buckle 232 has a buckle hole 233 for extension of the strip extension 22.

The patella protective member 3 is detachably attached to an inner side of the strip 2 for preventing a lateral movement of the patella, and includes an intermediate portion 31, two lateral portions 32, and a resilient cushion pad 33 attached to the intermediate portion 31.

The intermediate portion 31 has inner and outer faces 311, 312. The inner face 311 of the intermediate portion 31 is adapted to face the patella. The outer face 312 of the intermediate portion 31 is detachably fastened to an inner surface 211 of the patella bracing portion 21 by means of Velcro fasteners.

The two lateral portions 32 extend oppositely from two ends of the intermediate portion 31 and are fastened to the patella bracing portion 21. In this preferred embodiment, the two lateral portions 32 extend outwardly and respectively through the two holes 212 and are attached to the outer surface 312 of the patella bracing portion 21 by means of Velcro fasteners.

In the preferred embodiment, the resilient cushion pad 33 is disposed on the inner face 311 of the intermediate portion 31 and is adapted to cushion the patella. The resilient cushion pad 33 is made of an elastic material, for example, a foam material, a rubber, a thermoplastic elastomer, a silicone rubber, etc. Although the resilient cushion pad 33 is preferably disposed on the inner face 311, it may also be embedded in the intermediate portion 31. Besides, based on a desired cushion degree of the knee, the patella protective member 3 could be replaced with one that includes the resilient cushion pad 33 having a suitable elasticity, without replacing the whole patella support 200.

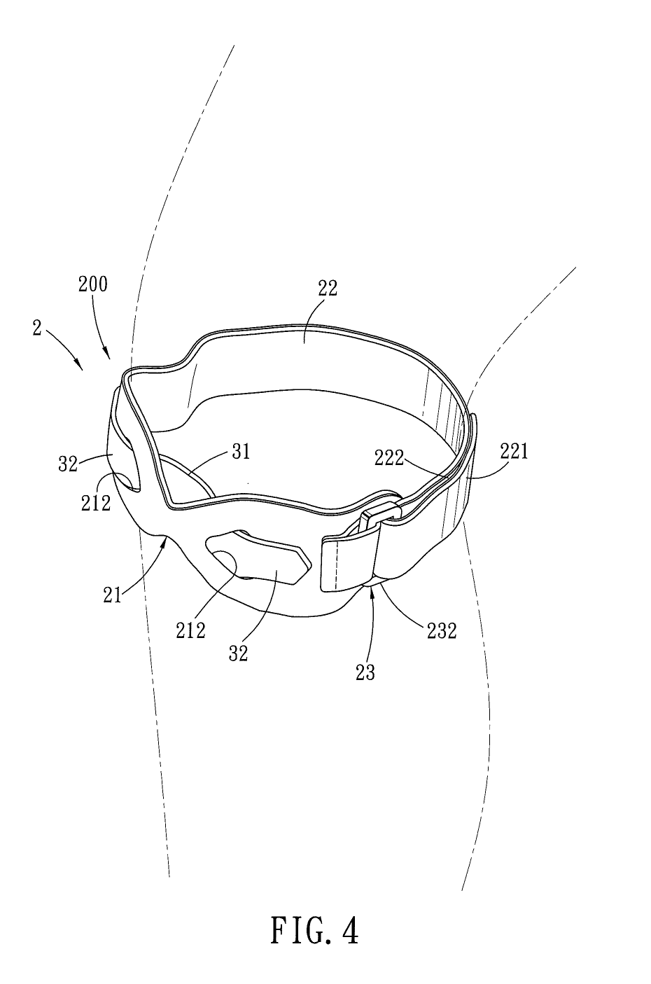

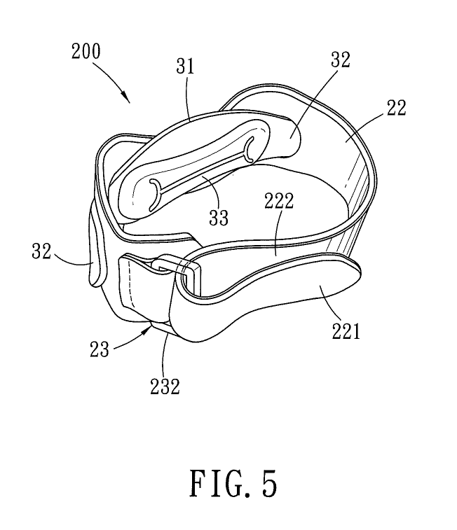

As shown in FIGS. 4 and 5, when wearing the patella support 200, the resilient cushion pad 33 is disposed to align with a lower portion of the patella, and the strip extension 22 is extended through the buckle hole 233. Thereafter, the strip extension 22 is folded back to connect the part 221 with the part 222 so that the strip 2 loops around the knee joint. Since the patella support 200 can adjustably brace and fit the patella, and since the resilient cushion pad 33 is disposed in the patella protective member 3 to cushion the patella, the user's patella can be supported and prevented from an abnormal motion or an injury. Furthermore, since the patella protective member 3 including the resilient cushion pad 33 is detachably attached to the strip 2, based on cushion requirements for different users, the patella protective member 3 can be replaced with one having a desired cushion degree.

While the present invention has been described in connection with what is considered the most practical and preferred embodiment, it is understood that this invention is not limited to the disclosed embodiment but is intended to cover various arrangements included within the spirit and scope of the broadest interpretations and equivalent arrangements.

What is claimed is:

1. A patella support, comprising:
a strip adapted to loop around a knee joint and including a patella bracing portion; and
a patella protective member attached to an inner side of said strip for preventing a lateral movement of a patella of the knee joint, and including:
an intermediate portion having an outer face fastened to an inner surface of said patella bracing portion, and an inner face adapted to face the patella;
two lateral portions extending oppositely from two ends of said intermediate portion, and fastened to said patella bracing portion; and
a resilient cushion pad attached to said intermediate portion and adapted to cushion the patella.

2. The patella support of claim 1, wherein said patella bracing portion is formed with two holes, said two lateral portions extending outwardly and respectively through said two holes and being attached to an outer surface of said patella bracing portion.

3. The patella support of claim 1, wherein said strip further includes a strip extension extending from one side of said patella bracing portion, and a fastener disposed on the other side of said patella bracing portion opposite to said strip extension, said strip extension being connected adjustably to said fastener such that said strip forms an adjustable loop.

4. The patella support of claim 3, wherein said fastener includes a buckle attached to an outer surface of said patella bracing portion and having a buckle hole for extension of said strip extension.

5. The patella support of claim 1, wherein said resilient cushion pad includes one of a foam material, a rubber, a thermoplastic elastomer, and a silicone rubber.

6. The patella support of claim 1, wherein said patella bracing portion has a narrow mid part, and two enlarged parts on two opposite sides of said narrow mid part, said two holes being disposed in said enlarged parts, respectively.
